(19) United States
(12) Patent Application Publication
(10) Pub. No.: US 2018/0132811 A1
(43) Pub. Date: May 17, 2018

(54) RADIOLOGICAL APPARATUS PARTICULARLY FOR VETERINARY USE

(71) Applicant: SKANRAY EUROPE S.R.L., San Lazzaro Di Savena (IT)

(72) Inventors: Luca PARENTI, San Lazzaro Di Savena (IT); Roberto MOLTENI, Caprino Veronese (IT); Nicola PETTAZZONI, Budrio (IT)

(21) Appl. No.: 15/807,871

(22) Filed: Nov. 9, 2017

(30) Foreign Application Priority Data
Nov. 11, 2016 (IT) 102016000113849

(51) Int. Cl. A61B 6/00

(52) U.S. Cl. CPC A61B 6/508; A61B 6/4405

(57) ABSTRACT

A radiological apparatus, particularly for veterinary use, includes a supporting base frame that defines a supporting surface for a patient to be radiographed, substantially radiolucent, and elements of obtaining radiographic images. The apparatus includes at least one assembly for emitting/receiving X-rays that can be moved manually with respect to the supporting base frame, along at least two directions of movement that are substantially perpendicular to each other and substantially parallel to the supporting surface. The emitter/receiver assembly further includes an X-ray generator supported by a head that is rigidly connected to a trolley situated below the supporting surface and supports a detector.

### CROSS REFERENCE TO RELATED APPLICATIONS

This application is related to and claims the benefit of Italian Patent Application No. 102016000113849, filed on Nov. 11, 2016, the contents of which are herein incorporated by reference in their entirety.

### TECHNICAL FIELD

The present disclosure relates to a radiological apparatus, particularly for veterinary use.

### BACKGROUND

As is known, the radiological apparatuses currently used to obtain radiological images of human or animal patients comprise, generally, the following components: an X-ray generator, with related power electronics to power it (i.e. a converter); a collimator for X-rays, with an illuminated device for centering the irradiated field; an electronic system for controlling the emission of X-rays; a radiolucent table for supporting the patient; mechanical structural elements, such as in particular the stand, for supporting and positioning the radiographic generator; and a radiographic detector and/or systems for accommodating/positioning it below the radiolucent table.

In conventional apparatuses, there may also be further subsystems and optional accessories, such as, for example, a radiographic grid with a Potter-Bucky device associated with it and, for apparatuses with a digital detector, optionally also the radiographic imaging software and patient records software.

In current practice, apparatuses as described above are commonly, if improperly, known as “bucky tables”.

In the simpler apparatuses of this type, such as, for example, the cheaper ones for veterinary radiology on small and medium-sized pets, the radiolucent table is typically constituted by a fixed support on which the animal patient is positioned and, as needed, moved by the operator, in order to make the irradiated field correspond to the anatomical area of interest.

Evidently, this method of working, in addition to being inconvenient, does not permit an adequate level of accuracy in the positioning of the animal, not least in consideration of the fact that the animal may not be inclined to cooperate.

In the vast majority of cases, however, and practically always in radiology on adult humans, the radiolucent table is floating i.e. it is installed on a lower base structure so as to be moveable, with respect to the base structure proper, along an axis parallel to its longitudinal extension and, often, also along an axis parallel to its transverse extension, so that the area to be radiographed can be located precisely in the irradiated field, which in turn is located at the underlying image detector.

Usually, the movement of the floating table (and the movement of any other elements that can be positioned) is electrically servo-actuated.

Radiological apparatuses provided with a floating table exhibit some drawbacks.

In fact, these apparatuses require that free space be left for the movement of the table at both longitudinal ends of the table proper, which entails the need to have a considerable space for the installation of the apparatus.

This is not very important for hospital radiology rooms, which are generally quite large, but it becomes a problem in small clinics, especially veterinary clinics, which are often housed in residential buildings where space is often limited.

To this must be added as well the need to properly dimension the resistance to cantilevered mechanical load of the table, with consequent costs, also taking account of the high safety standards required.

Another disadvantageous aspect of radiological apparatuses with a bucky table, especially in veterinary applications, arises from the fact that generally the actual radiographic emission is preceded by a step of preparation. Such step of preparation can imply, among other things, actuating or achieving rotation speed in generators that use a rotating anode X-ray tube, reaching the temperature for thermionic emission by the filament of the X-ray tube, readying electronic devices, such as for example flushing dark charges in image detectors, as well as operations to ready the software, for example for the data transmission of the digital image.

The step of preparation can require several seconds, during which time the patient should remain still without moving even slightly from the position in which it has been arranged. Typically, the sequence of steps of preparation and of subsequent emission of X-rays is generally commanded by a two-stage manual button. In particular, actuation of the first stage of the button begins the step of preparation, the completion of which is indicated by way of an acoustic and/or luminous signal, while the actual emission is then triggered by actuating the second stage of the button.

The step of preparation is particularly disadvantageous in veterinary radiology, since the animal patient will not be cooperating and the operator therefore needs the X-ray emission to happen immediately after actuating the emission command, which is carried out by the operator when the operator considers that the animal is ready and before the animal might start moving. Among other things, the step of preparation may imply stimuli, such as for example the buzzing of the anode accelerating in rotation, which disturb the animal and cause it to move.

### SUMMARY

The aim of the present disclosure is to provide a radiological apparatus particularly for veterinary use which is capable of improving the known art in one or more of the above mentioned aspects.

Within this aim, the disclosure provides a radiological apparatus particularly for veterinary use that makes it possible to keep the patient animal in a fixed position without needing to have to move it in order to position the area to be radiographed under the irradiated field.

The disclosure also provides a radiological apparatus particularly for veterinary use that does not require free space at the sides of the table for supporting the patient.

The present disclosure further provides a radiological apparatus particularly for veterinary use that does not require onerous dimensioning of the mechanical structures.

The present disclosure develops a radiological apparatus that enables the operator to obtain the radiographic emission practically instantaneously upon actuating the emission command

Furthermore, the present disclosure also overcomes the drawbacks of the known art in a different manner to any existing solutions.

The disclosure further provides a radiological apparatus particularly for veterinary use that, in addition to offering the widest guarantees of reliability and safety during its operation, is structurally simple, so as to be easy to make and also highly competitive from a purely economic viewpoint.

These advantages and features which will become better apparent hereinafter are achieved by providing a radiological apparatus particularly for veterinary use according to claim 1, optionally provided with one or more of the characteristics of the dependent claims.

### BRIEF DESCRIPTION OF THE DRAWINGS

Further characteristics and advantages of the disclosure will become better apparent from the detailed description that follows of a preferred, but not exclusive, embodiment of the radiological apparatus particularly for veterinary use according to the disclosure, which is illustrated for the purposes of non-limiting example in the accompanying drawings wherein:

FIG. 1 is a partially transparent perspective view of the apparatus according to the disclosure;

FIG. 2 is a perspective view from below of the apparatus according to the disclosure;

FIG. 3 is a partially cross-sectional side view of the apparatus according to the disclosure;

FIG. 4 is a partially transparent plan view from above of the apparatus according to the disclosure;

FIG. 5 is a partially cross-sectional plan view from above of the apparatus according to the disclosure with parts shown transparent; and

FIGS. 6 and 7 are partially transparent plan views from above of the apparatus according to the disclosure with an assembly for emitting/receiving X-rays in two different positions with respect to a base frame of the apparatus.

### DETAILED DESCRIPTION OF THE DRAWINGS

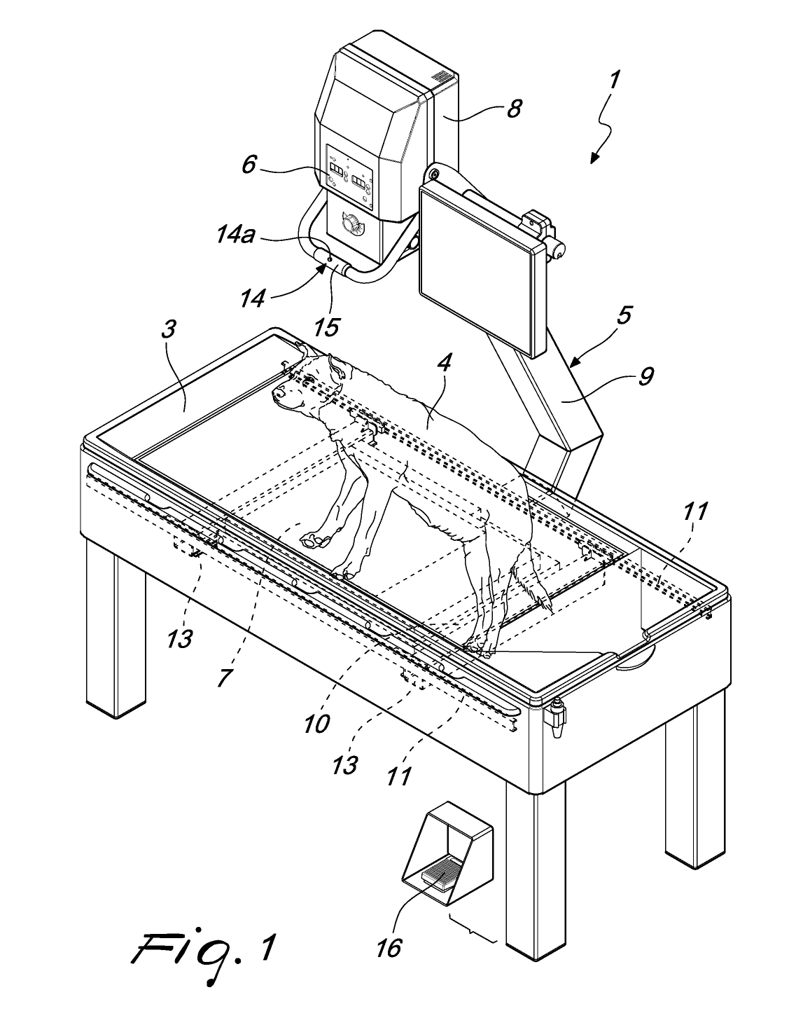

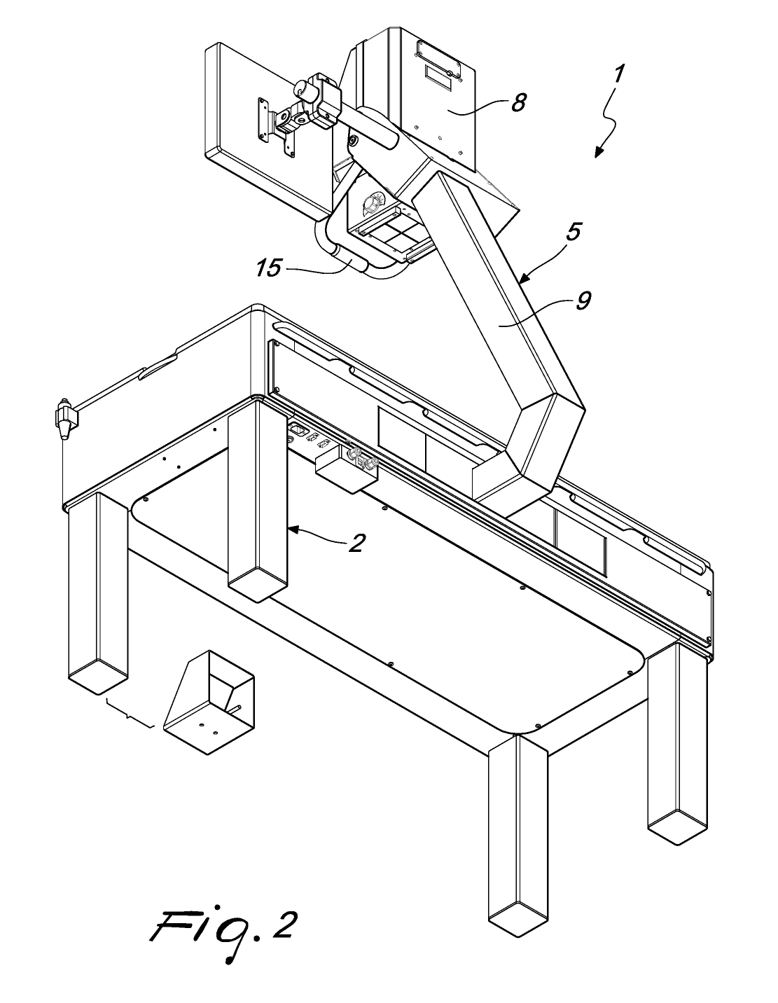

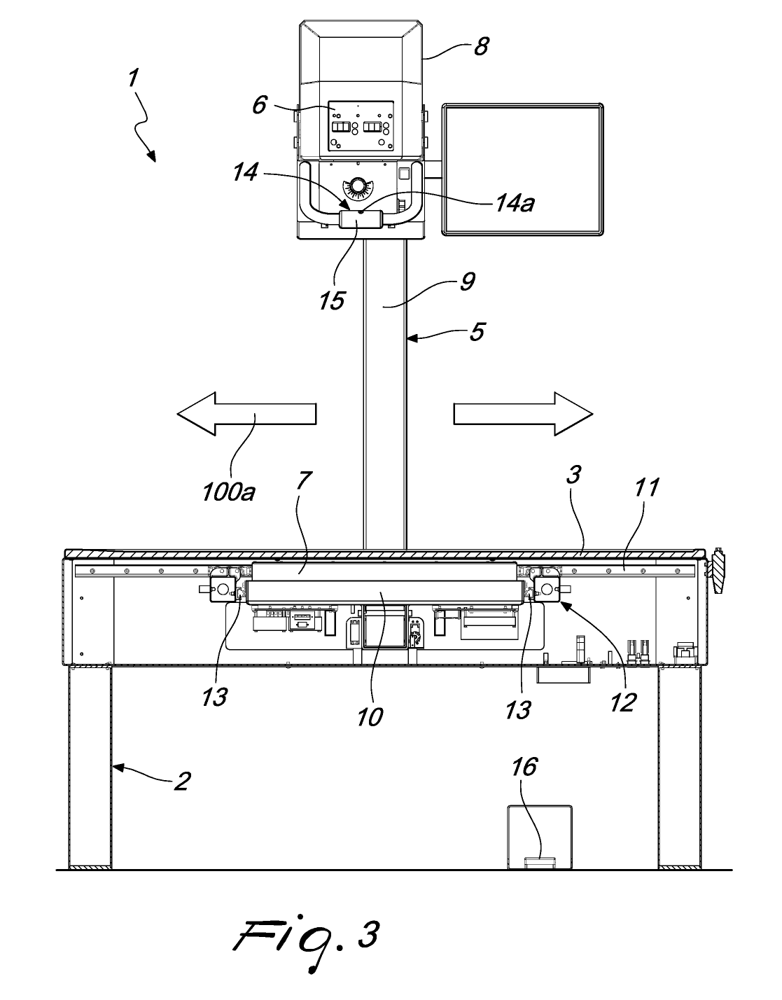

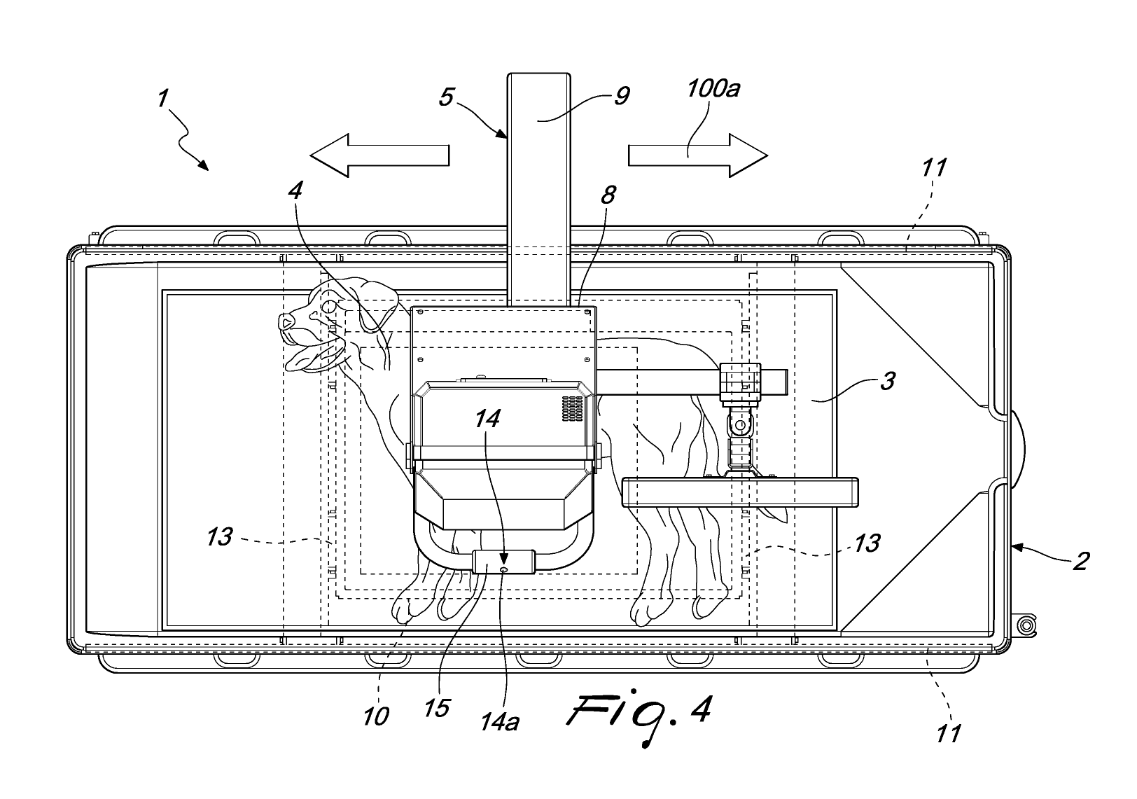

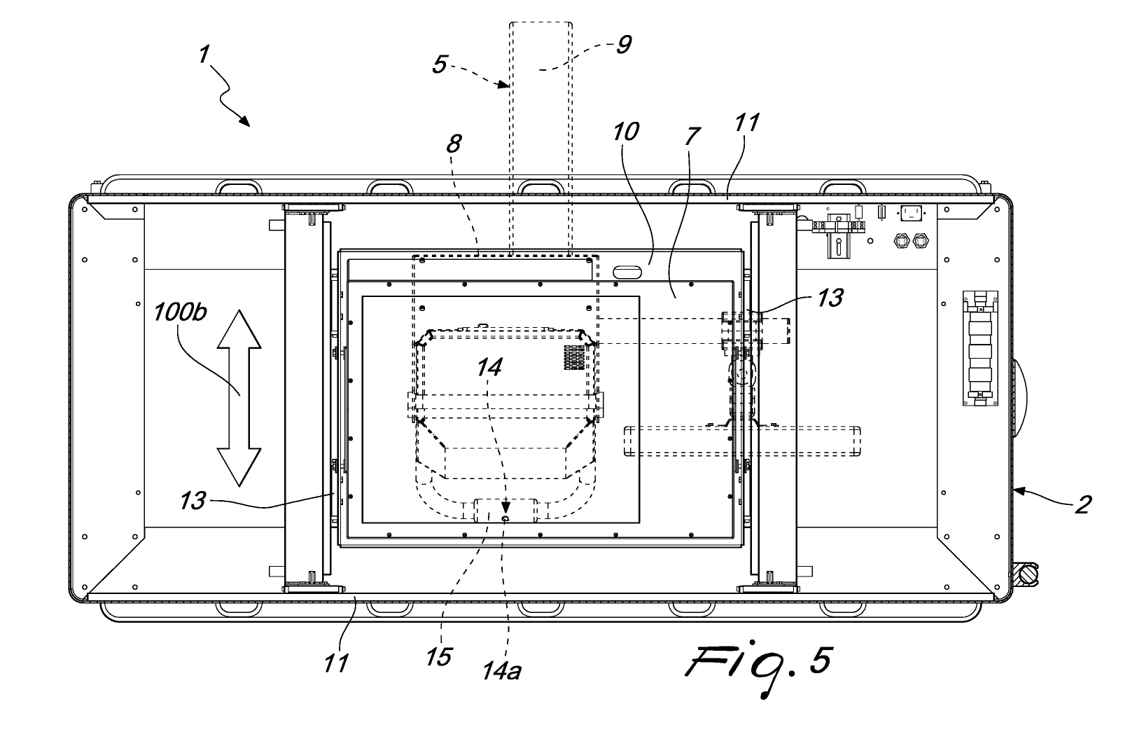

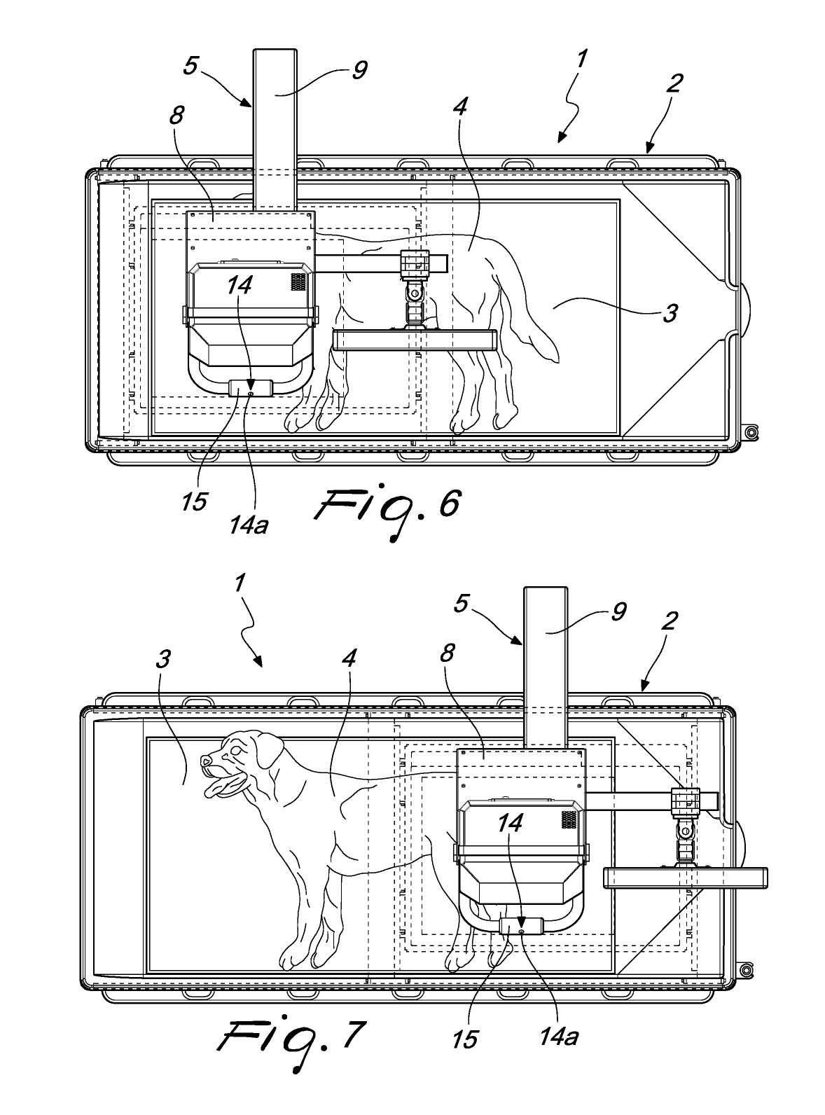

With reference FIGS. 1-7, the radiological apparatus, particularly for veterinary use, according to the disclosure, generally designated by the reference numeral 1, comprises a base frame 2 that defines a supporting surface 3 for the animal patient 4 to be radiographed.

The apparatus is further provided with means of obtaining radiographic images, which comprise at least one assembly 5 for emitting/receiving X-rays.

Advantageously, the means of obtaining radiographic images can further comprise, for the purposes of example, an X-ray collimator, with an illuminated device for centering the irradiated field, at least one electronic emission control unit, a radiographic imaging system with special management software and other, conventional components.

The emitter/receiver assembly 5 can be moved manually by the user, with respect to the base frame 2, along at least one direction of movement that is substantially parallel to the supporting surface 3.

In this manner, the animal 4 can remain arranged on the supporting surface 3 without needing to be moved by the user, it being instead possible, with manual movement, to move the emitter/receiver assembly 5, with respect to the base frame 2 and, therefore, with respect to the supporting surface 3, so as to be able to position the irradiated field at the anatomical region to be radiographed.

With this arrangement, space requirement, in plan view on the floor, by the apparatus can be limited to the mere physical dimensions of the base frame 2, without requiring additional free space at the sides, as with conventional apparatuses that use a floating table. Furthermore, again by virtue of such structural organization, for the same patient weight, the dimensioning of the base frame 2 and of the supporting surface 3 is much less onerous with respect to a traditional floating table, which instead has to be supported in a cantilever fashion on its underlying base frame.

Conveniently, the emitter/receiver assembly 5 can be moved manually, with respect to the base frame 2, at least along a direction of movement that is substantially parallel to the longitudinal extension of the supporting surface 3.

According to the disclosure, the emitter/receiver assembly 5 can be moved manually by the user, with respect to the base frame 2, along at least two directions of movement 100a, 100b that are substantially perpendicular to each other and substantially parallel to the supporting surface 3, conveniently both in one direction and in the opposite direction.

Delving deeper into the details, the emitter/receiver assembly 5 comprises, conveniently, an X-ray generator 6 and a radiographic detector 7, which are integrally mutually connected and are mounted so that they can move on the base frame 2 along the aforementioned directions of movement 100a, 100b.

According to the illustrated example, the supporting surface 3 is substantially radiolucent and the generator 6 is arranged so as to face the supporting surface 3, while the detector 7 is situated on the opposite side of the supporting surface 3 with respect to the generator 6.

Advantageously, the generator 6 is supported by a head 8, spaced apart from the supporting surface 3 in an upper region and rigidly connected, by way of at least one connecting arm 9, to a trolley 10 that is situated below the supporting surface 3 and supports the detector 7.

In particular, the trolley 10 is slideably supported by the base frame 2 along the directions of movement 100a and 100b.

Conveniently, the trolley 10 can be slideably mounted on guiding means that can move with respect to the supporting frame 2, which extend along a direction that is substantially parallel to one of the directions of movement 100a, 100b and which are slideably mounted along guiding means that are fixed with respect to the supporting frame 2, which extend along a direction that is substantially parallel to the other direction of movement 100a, 100b.

In more detail, the base frame 2 supports, below the supporting surface 3, at least one first sliding linear guide 11, which extends substantially parallel to a first direction of extension of the supporting surface 3, for example a direction of longitudinal extension.

Such first linear guide 11 is slideably engaged by a slide 12, which is substantially constituted by at least one second sliding linear guide 13, which extends substantially parallel to a second direction of extension of the supporting surface 3, which is substantially perpendicular to the first direction of extension, for example a transverse direction of extension, and by the trolley 10 which, in its turn, slideably engages the second sliding linear guide 12.

More preferably, there is a pair of first linear guides 11 arranged on mutually opposite sides with respect to the slide 12 and a pair of second linear guides 13 that are mutually opposite with respect to the trolley 10.

Advantageously, the detector 7 may be digital, and in particular it electronically transmits the digital image practically in real-time, i.e. not delayed. Therefore, the detector 7 does not require, at each radiographic exposure, a cassette with radiographic film or with a photostimulable storage phosphor (PSP) screen to be extracted from its receptacle for developing or scanning in a separate station and it can, therefore, be installed in the supporting frame 2, without requiring physical access by the user in normal operation.

Advantageously, likewise means of automatic activation of the step of preparation of the means of obtaining radiographic images upon the manual displacement of the emitter/receiver assembly 5, with respect to the base frame 2, along the directions of movement 100a, 100b may be provided.

Such step of preparation, which precedes the actual emission of X-rays by the generator 6, can, for example, entail, among other things, switching on the centering/collimation light, preparing the X-ray tube, readying other possible electronic and software operations, etc.

In this manner, upon completion of the positioning of the emitter/receiver assembly 5 by the user operator, by way of the manual displacement thereof with respect to the supporting frame 2, along the directions of movement 100a, 100b, the means of obtaining radiographic images can be ready to operate and, as a consequence, when the operator triggers the radiographic emission, this can happen practically instantly, without waiting times.

Advantageously, there are disengageable means of locking the emitter/receiver assembly 5 in place with respect to the base frame 2, which are constituted, for example, by braking means that are adapted to prevent the sliding of the trolley 10 along the second guides 13 and of the slide 12 along the first guides 11. Such disengageable locking means are functionally connected to command means 14, which can be actuated by the user to command the deactivation of the disengageable locking means and therefore allow the displacement of the emitter/receiver assembly 5 with respect to the base frame 2.

According to a preferred embodiment, the step of preparation of the means of obtaining radiographic images is activated automatically following the actuation by the user of the command means 14 that make it possible to deactivate the disengageable locking means of the emitter/receiver assembly 5.

Conveniently, the emitter/receiver assembly 5 is associated with a handle 15, which is engageable manually by the user in order to cause the manual displacement of the emitter/receiver assembly 5, with respect to the base frame 2, along the directions of movement 100a, 100b.

The command means 14 comprise, advantageously, an actuation device 14a, positioned on the handle 15. The actuation device 14a can, for example, be constituted by an element that can be maneuvered, such as, for example, a lever, a button or the like, or by a pressure or proximity sensor, capable of detecting the presence of the hand of the user on the handle 15.

It should be noted that the activation of the generator 6 can be, conveniently, triggered by the user by way of a single-stage actuation, such as, for example, a pedal 16 arranged proximate to the base frame 2.

Operation of the apparatus according to the disclosure is the following.

The operator positions the animal patient 4 on the supporting surface 3 and with one hand grasps the handle 15 and actuates the command means 14, so as to deactivate the disengageable locking means of the emitter/receiver assembly 5 with simultaneous automatic activation of the step of preparing the means of obtaining the radiographic images.

Subsequently, the operator manually moves, with respect to the base frame 2, the emitter/receiver assembly 5 along one or both of the directions of movement 100a, 100b, until the emitter/receiver assembly 5 is brought to the position that makes it possible to irradiate the anatomical region to be radiographed.

Once the emitter/receiver assembly 5 is positioned correctly, the operator can actuate the pedal 16 to trigger the emission of X-rays by the generator 6.

Such emission can occur practically instantly upon actuation of the pedal 16 by the operator, since the step of preparation that was automatically activated previously has been concluded, in the meantime, during the step of positioning the emitter/receiver assembly 5.

The detector 7, struck by the X-rays, will send its signal to the radiographic imaging system which, in turn, will proceed to process the radiographic images.

In practice it has been found that the disclosure fully achieves the intended advantages and features, by providing a radiological apparatus, particularly for veterinary use, that makes it possible to avoid moving the animal patient on the supporting surface, which does not require maneuvering space around it, so that it can be easily installed even in relatively small rooms, and which is simpler in construction than apparatuses with floating tables.

All the characteristics of the disclosure, indicated above as advantageous, convenient or similar, may also be missing or be substituted by equivalent characteristics.

The individual characteristics set out in reference to general teachings or to specific embodiments may all be present in other embodiments or may substitute characteristics in such embodiments.

The disclosure thus conceived is susceptible of numerous modifications and variations.

Thus for example, with the same concept, a radiological apparatus can also be provided for pediatric human patients, both newborn infants and children.

Moreover, all the details may be substituted by other, technically equivalent elements.

In practice the materials employed, provided they are compatible with the specific use, and the contingent dimensions and shapes, may be any according to requirements and to the state of the art.

1. A radiological apparatus comprises a supporting base frame that defines a supporting surface for a patient to be radiographed, substantially radiolucent, and means of obtaining radiographic images, which comprise at least one emitter/receiver assembly configured for emitting/receiving X-rays that can be moved manually with respect to said supporting base frame along at least two directions of movement that are substantially perpendicular to each other and substantially parallel to said supporting surface, said emitter/receiver assembly comprising an X-ray generator and a radiographic detector connected integrally to each other, said X-ray generator being arranged facing said supporting surface, said detector being arranged on the opposite side of said supporting surface with respect to said X-ray generator, said X-ray generator being supported by a head spaced apart from said supporting surface in an upper region and rigidly connected, by way of at least one connecting arm, to a trolley situated below said supporting surface and supporting said detector, said trolley being slideably mounted on guiding means that can move with respect to said supporting frame, which extend along a direction substantially parallel to one of said directions of movement and are slideably mounted along guiding means that are fixed with respect to the supporting frame, which extend along a direction substantially parallel to the other direction of movement.

2. The radiological apparatus according to claim 1, wherein said supporting base frame supports, below said supporting surface, at least one first sliding linear guide extending substantially parallel to a first direction of extension of said supporting surface, said at least one first linear guide being slideably engaged by a slide that comprises at least one second sliding linear guide extending substantially parallel to a second direction of extension of said supporting surface which is substantially perpendicular to said first direction of extension, said at least one second sliding linear guide being slideably engaged by said trolley.

3. The radiological apparatus according to claim 1, further comprising means of automatic activation of a step of preparation of said means of obtaining radiographic images upon the manual displacement of said emitter/receiver assembly, with respect to said supporting base frame, along said at least two directions of movement.

4. The radiological apparatus according to claim 1, further comprising disengageable locking means for locking said emitter/receiver assembly in place with respect to said supporting base frame and command means for the deactivation of said disengageable locking means.

5. The radiological apparatus according to claim 3, wherein said step of preparation of said means of obtaining radiographic images is automatically activated following the actuation of said command means by the user.

6. The radiological apparatus according to claim 4, further comprising a handle associated with said emitter/receiver assembly and engageable manually by the user in order to cause the manual displacement of said emitter/receiver assembly, with respect to said supporting base frame, along said at least two directions of movement, said command means comprising an actuation device positioned at said handle.
